(19) United States
(12) Patent Application Publication
(10) Pub. No.: US 2019/0167592 A1
(43) Pub. Date: Jun. 6, 2019

(54) PROCESS FOR DIRECTLY COMPRESSIBLE CO-PROCESSED EXCIPIENT FOR MODIFIED RELEASE APPLICATION

(71) Applicant: GANGWAL CHEMICALS PRIVATE LIMITED, Mumbai (IN)

(72) Inventors: Anil K. Jain, Mumbai (IN); Rajan KALE, Mumbai (IN)

(21) Appl. No.: 16/317,463

(22) PCT Filed: Jul. 5, 2017
(86) PCT No.: PCT/IB2017/054044

§ 371 Date: Jan. 11, 2019

(30) Foreign Application Priority Data
Jul. 13, 2016 (IN) 201621023988

(51) Int. Cl. A61K 9/16; A61K 47/38; A61K 47/32; A61K 47/02; A61K 47/12; A61K 31/196; A61K 31/155; A61K 31/4178; A61K 31/137

(52) U.S. Cl. CPC A61K 9/1694; A61K 47/38; A61K 47/32; A61K 47/02; A61K 47/12; A61K 9/1652; A61K 31/137; A61K 9/1611; A61K 9/1617; A61K 31/196; A61K 31/155; A61K 31/4178; A61K 9/1635

(57) ABSTRACT

The present invention provides a process for manufacturing a co-processed directly compressible excipient for sustained/extended release formulation based on pharmaceutically acceptable inert diluent, hydrophilic swellable polymer and a binder.

### RELATED APPLICATION

This application is related to and takes priority from Indian Provisional Application 201621023988 filed with Mumbai Patent Office on 13 Jul. 2016, and is incorporated herein in its entirety.

### FIELD OF THE INVENTION

The present invention is related to a process of manufacturing a co-processed directly compressible excipient for sustained/extended release formulation based on pharmaceutically acceptable inert diluent, hydrophilic swellable polymer and a binder.

### BACKGROUND

Tablet formulations are especially critical when the composition consist of at least three components such as pharmaceutically active components having wide particle size distributions, directly compressible excipients and optionally additional auxiliary substances.

Excipients which are suitable for direct compression must have sufficient plastic deformability and good flow properties and should not exhibit any segregation tendency.

Advantage of direct compression is that granulation of the pharmaceutically active component is not required and thus moisture/solvent sensitive components can also readily and easily be processed.

Use of direct compression is improves overall stability of dosage forms. Exposure of API and formulation to moisture, solvents and heat is eliminated by direct compression. Incompatibilities arising due to moisture, solvent and heat are thus evicted.

Another advantage of direct compression is that it is economical since the process requires fewer unit operations. This means less equipment, lower power consumption, less space and less inventory, less time and less labour leading to reduced production cost. During manufacturing fewer unit operations are involved, hence human handling and exposure to dosage forms is also minimized.

API handling and processing steps are reduced, so overall API losses are reduced.

Hence, there is a great demand for excipients which can be simply mixed with the range of pharmaceutical active component, either single or combinations and optionally with additional tabletting excipients (if necessary) and subsequently be directly compressed.

Directly compressible excipient properties described above are in most cases not achieved by simple physical mixing of commercially available individual components. Processed or co-processed granulates comprising different desired functional compression excipients are therefore desired.

Spray drying in cold water (U.S. Pat. Nos. 4,744,987, 8,932,629), spray drying in hot water (WO1995020328), hot melt granulation (US20140275242, US20100120723), high shear mixer granulator (WO2000004880) are some of the technologies mentioned in prior arts for manufacturing directly compressible excipients.

It is therefore desirable to provide tabletting excipients, in particular, directly compressible excipients that have superior properties of tablet formulations with regard to release behavior and/or compressibility. These properties will further contribute to improvement of the tablet profile in terms of the following criteria: tablet hardness, friability resistance, and release profile modification and/or compression force-hardness profile.

The present invention pre-optimizes conditions to provide directly compressible co-processed excipient having improved flow properties and optimized particle size and narrow size distribution which may be mixed with a wide range of active pharmaceutical agents and directly compressed into solid dosage forms, more specifically, matrix type of tablets. The invention thus avoids routine pharmaceutical dry or wet granulation process, binders, fillers, disintegrants, glidants and so on, used for the purpose of tableting at the user end.

### BRIEF DESCRIPTION OF DRAWINGS

FIG. 1. Dissolution Profile of Diclofenac Sodium extended release tablets

FIG. 2. Dissolution Profile of Metformin Hydrochloride extended release tablets

FIG. 3. Release profile of Losartan potassium extended release tablets

FIG. 4. Release profile of Tramadol hydrochloride extended release tablets

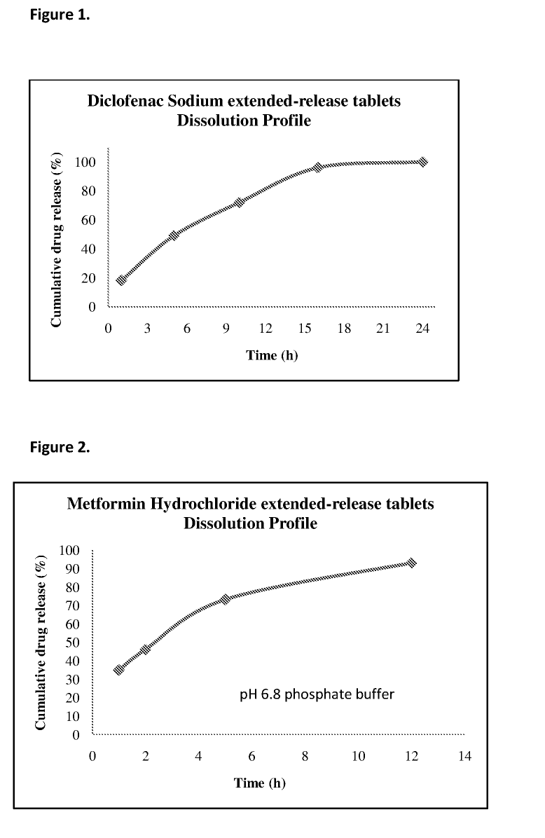

### SUMMARY OF THE INVENTION

The present invention provides a process for producing a co-processed directly compressible excipient that can be used as tabletting excipient further which can be directly compressed into solid dosage forms, preferably tablets, more specifically matrix type tablets.

In one aspect, the invention provides a drug release rate-controlling excipient which enables improved control over active ingredient release from tablets when compared to the individual conventional rate controlling excipients without otherwise compromising the tablet properties.

In another aspect, the present invention provides a process to prepare and manufacture the co-processed excipient having improved flow properties, improved compaction performance such as improved tablet hardness, tablets having smooth surface and reduced friability, all of which contribute to direct compressible solid dosage forms, with or without the need of any auxiliary excipients.

### DETAILED DESCRIPTION

Various characteristics of the particulate co-processed excipients of the present invention, for example particle size, particle size distribution, moisture content, bulk density, with regard to granulation process is described in detail below, to enable preparation and manufacturing. These physical characteristics are in large measure dependent on the manner in which the components of this particulate excipient are co-processed. It is for this reason that the wet granulation process and drying step in the co-processing procedure is especially critical.

In one embodiment, the present invention provides a process for preparation and manufacturing of directly compressible co-processed excipient which contains a combination of ingredients in preselected proportions which provides a desired slow/modified release profile and better tabletting performance for oral solid dosage forms especially matrix tablets for wide variety of drugs.

The present invention provides a process for producing a directly compressible granulate comprising the following sequential steps:

Step 1: Wetting of suitable diluent with water to form wet mass;

Step 2: Unload wet mass of step 1 and collect in collector;

Step 3: Load previously sifted (sieve size: 60#) suitable release retarding hydrophilic swellable polymers in high shear mixer granulator having impeller—chopper combination and dry mix for 5 min (dry blending);

Step 4: Slow addition of wet mass of suitable diluent (prepared in step 1) to the dry powder blend of step 3 to granulate this dry blend.

Step 5: Addition of extra water if required during granulation;

Step 6: Discharging wet granulate mass and drying it in fluidized bed dryer (60° C.);

Step 7: Addition of glidant and lubricants to the dried and sized granules.

In the present invention, the terms ‘directly compressible granulate’ and ‘directly compressible excipient’ are used interchangeably.

Suitable diluent/filler provide the required bulk of the tablet when the quantity of active pharmaceutical ingredient itself is inadequate to produce tablets of adequate weight and size and strength. Suitable diluent selected should be non-reactive with the drug substance and moreover it should be compatible with other excipients or composition of the formulation in which it is incorporated. It should not exert any physiological or pharmacological activity of its own. It should be inert material having consistent physical and chemical characteristics. It should neither promote nor contribute to segregation behavior of the granulation or powder blend to which they are added. It should be able to be milled (size reduced) if required. It should neither support microbiological growth in the dosage form nor contribute to any microbiological load. It should neither adversely affect the performance of the dosage form such as dissolution of the product nor interfere with the bioavailability of active pharmaceutical ingredient. Colorless or nearly so materials are preferred.

Carbohydrate substances such as sugars, starches and celluloses functions as diluents when incorporated in direct compression system.

In a preferred embodiment water insoluble diluent is preferred.

Microcrystalline cellulose (MCC) is a purified, partly depolymerized cellulose with shorter, crystalline polymer chains. In the present invention, MCC primarily serves as a diluent. It's strong binding performance also makes MCC one of the most commonly used and suitable filler and binder in drug formulations.

In yet another embodiment the present invention allows easy adjustment of particle size and flow properties of granulates as per requirement.

Granulates of co-processed directly compressible excipient obtained by the present invention have desired flow properties, bulk density, narrow particle size distribution, moisture content <5%, improved hardness and reduced friability for tablets, better swelling behavior of the placebo matrices, when directly compressed as compared to the physical mixtures compressed at same compression force/pressure, of the same composition.

A modified or extended release of pharmaceutical active ingredient(s) from dosage forms, can be accomplished by homogeneously dispersing the active ingredient(s) in a matrix system of co-processed directly compressible excipient prepared using diluent(s) and hydrophilic swellable polymer. Wherein the substantially neutral polymer is selected from the group of synthetic polymers (poly acrylic acid derivatives, poly vinyl alcohol derivatives etc. and other), cellulose based polymers (nonionic, substituted etc. and other), hydrocolloids (gum arabic, guar gum, xanthan gum, alginic acid derivatives, carrageenan, chitosan etc. and others), polymers or any suitable combination thereof.

In preferred embodiment hydrophilic swellable polymer is from cellulose based polymers. Cellulose is preferably obtained from natural sources. Cellulose based polymers are chemically modified celluloses in which the hydroxyl groups are at least partially alkylated, hydroxyl-alkylated, sulfonated, nitrated, carboxyl-alkylated or/and xanthogenated independently of one another.

Cellulose based polymers are preferably used as hydrophilic swellable polymer due to their good swelling properties, better gel layer strength and good compressibility. Examples of these polymers are hypromellose [hydroxypropylmethyl cellulose (HPMC)], hypromellosepthalate, hydroxypropyl cellulose (HPC), hydroxyethyl cellulose (HEC), carboxymethyl cellulose (CMC), carboxyethyl cellulose (CEC), methyl cellulose (MC), ethyl cellulose (EC) as well as salts (sodium or/and calcium salts) and/or mixtures, or any cellulose derivative or a combination thereof. Hypromellose (HPMC), ethyl cellulose (EC), hydroxypropyl cellulose (HPC) and hydroxyethyl cellulose (HEC) are preferred and more preferably hypromellose (HPMC) is used.

Water or organic solvents or starch paste or hydrophilic polymer solutions or water insoluble cellulose based wet mass and or mixtures thereof, for example, are used as binder. Suitable organic solvents are for example methanol or ethanol or isopropyl alcohol or acetone or mixtures of thereof. Mixtures of solvents are also used. Water or alcohol and/or mixtures thereof are preferably used as the binder in first step to form wet mass of water insoluble diluent (as detailed previously). Water is a particularly preferred as binder. In order to produce the solution and/or wet mass, the diluents [starch or MCC or optionally cellulose (derivative)], alone or mixtures thereof, are incorporated into at least one liquid or binder for example while stirring mechanically. Standard stirring devices are used for the incorporation.

The present alternatively provides starch paste and hydrophilic swellable polymeric solution that can be used as binder.

The weight ratio between binder and other components is for example between 0/100 to 95/5, preferably between 0/100 to 10/90, preferably between 0/100 to 30/70 and more preferably 0/100 to 50/50.

Powder blends other than binder and starting material are dry mixed in next step were mixed at medium impeller speed of 50-150 rpm, preferably 70-130 and more preferred speed is 90-110 rpm. High shear mixer granulator having impeller—chopper combination is preferred in the present invention.

In further steps dry blend is preferably wet granulated using the wet mass prepared using starting materials such as inert diluents like but not limited to microcrystalline cellulose or starch or optionally mixture of microcrystalline cellulose with other cellulose derivative or any suitable starch derivative.

In this invention the binder liquid is at least partially or completely removed from the granulates by suitable drying processes such as tray drying, vacuum drying and any other suitable method employed for the drying process known to the person skilled in the art. Faster drying of granulates is preferred. Preferred drying method is one in which wet mass is dried using current of hot air supplied from bottom part of bowl with specific velocity. Hot air entering into the bowl of dryer will fluidize the bed of wet mass and accelerate the drying process. Air for fluidization is heated to the temperature to the extend such that it will not affect the stability of wet mass or the individual components employed in the present invention. Preferred range of temperature for drying is 40-100 degree Celsius, more preferably from 55-75 degree Celsius. Preferred type of dryer is fluidized bed dryer.

Content of free liquid, preferably water, in dried granulate is <8% by weight, preferably <6% by weight and more preferably <5% by weight based on the total mass of granulate.

The granulate particles obtained are preferably spherical or spheroid shaped. Such a morphology is advantageous for the flow properties of granulate.

Furthermore, the present invention provides a composition which comprises the granulate, which can be used to modulate the drug release of at least one pharmaceutically active component or more than one pharmaceutically active component preferably in combination. The weight ratio of granulate to pharmaceutically active component can vary within any ranges and is between 99.9% and 5%, preferably between 99% and 30% and more preferably between 99% to 50%.

Suitable excipients are, for example, but not limited to, lubricants and/or glidants such as stearic acid, magnesium stearate, calcium stearate, sodium stearate, boric acid, sodium lauryl sulfate, magnesium lauryl sulfate, corn starch, talcum, flow regulator and hardness enhancing agent such as silicon dioxide, hydrated sodium silico-aluminate can be used as auxiliary excipients.

The outcome or end product obtained by the process described in the present invention substantially overcome the need for conducting further experimentation needed to optimize tabletting properties for a particular therapeutically active medicament.

Upon oral ingestion and contact with gastric fluid the slow release matrix tablets prepared using the co-processed excipient prepared according to process of the present invention swells and gels to form a protective gel layer and swollen matrix from which the drug is released.

The combination of the hydrophilic swellable polymer with the inert diluent and lubricants provide a ready to use matrix forming excipient in which a formulator need only to blend the desired active medicament and then directly compress the blend to form slow release tablets.

The average particle size of the co-processed directly compressible excipient obtained from the present invention ranges from about 200 microns to about 400 microns and particle size distribution ranges from 50 microns to about 600 microns. The average particle size and size distribution of co-processed directly compressible excipient obtained using process described in the present invention be such that it will be possible to form pharmaceutically acceptable tablets upon direct compression, without segregation in pre-compression blends.

The desired bulk and tapped densities of the granulation of the present invention are normally between 0.3 to about 0.8 g/mL, with an average density of about 0.4 to about 0.7 g/mL.

The average flow rate of the granulates prepared in accordance with the present invention are from about 5 g/sec to about 3 g/sec.

Examples given below further aid in the understanding of the invention. However, no aspect of the example should be construed as limiting the scope of the invention.

### EXAMPLES

### Example 1

Co-processed directly compressible slow release excipient according to the process described in present invention is prepared as follows. First, microcrystalline cellulose (10-20%) and water are mixed in high shear (rapid mixer) granulator having impeller-chopper combination for 10 minutes to form wet mass, in 1:2 ratio. Unload this mixture from granulator. Load hydroxypropyl methyl cellulose (60-80%), ethyl cellulose (0.5-5%), hydroxypropyl cellulose (1-5%) in rapid mixer granulator. Dry blend this mixture for 5 minutes. After dry blending add wet mass of microcrystalline cellulose to the dry blend and granulate it. Add extra water if required for granulation. The mixed product, which is now in the form of granules, is removed from the granulator and dried in a fluidized bed dryer at a temperature of about 60° C.-70° C. to a loss on drying weight between 2-5%.

The dried granulates is then passed through a 20 mesh screen. Screened granulate lubricated using magnesium stearate (0.5-2.0%) and silicon dioxide (0.5-2.0%) is added as anti-adhering agent and flow promoter. The product is now ready to be used as a slow release excipient which is suitable for direct compression with any active medicament to form a slow release tablet.

### Example 2

Co-processed directly compressible slow release excipient according to the process described in present invention is prepared as follows.

Binding agent is prepared by mixing starch paste (5-12%) and aqueous solution of hydroxypropyl cellulose (0.5-4%) in a ratio of 5:1. This binder mixture is passed through 12 mesh screen to make it homogenous. Hydroxypropyl methyl cellulose (40-75%), ethyl cellulose (0.5-5%), microcrystalline cellulose (20-45%) were individually sifted through 60 mesh screen. Sifted materials were loaded to the rapid mixer granulator for dry mixing for 5 min. While running impeller and chopper, binding agent was added to the dry blend and granulated for 10 minutes. The granulates were then dried in fluidized bed dryer at 60° C. to a loss on drying weight between 2-5%. The dried granulates is then passed through a 20 mesh screen. Screened granulate lubricated using magnesium stearate (0.5-2.0%) and silicon dioxide (0.5-2.0%) is added as anti-adhering agent and flow promoter.

Granulates were tested for flow properties and other physical characteristics. In addition, particle size and size distribution pattern (data not shown) were also determined. The results are shown in Table 3.

TABLE 3
Physical properties of Example 1 and Example 2

| Properties | Example 1 | Example 2 |
|---|---|---|
| Flow rate (g/second) | 4.47 | 3.33 |
| Angle of repose (°) | 37.19 | 41.24 |
| Bulk density g/mL) | 0.4176 | 0.39 |
| Tap density (g/mL) | 0.5286 | 0.50 |
| Loss on drying (%) | 1.03 | 2.116 |
| Average particle size (μ) | 200-400 | 200-400 |

Release Retarding Potential of Co-Processed Directly Compressible Excipient of Present Invention

Product obtained by following the process described in present invention was evaluated for release retarding potential. Tablets were prepared as shown in Tables 4 and 6. Prepared tablets were tested for dissolution test as per pharmacopoeia dissolution methods. The data is represented as percentage of drug released versus time (hours). The results are provided in Tables 5 and 8.

### Example 3. Diclofenac Sodium Extended Release Tablets—100 mg

TABLE 4
Formulation of diclofenac sodium extended release tablets

| Sr. No. | Name of Ingredients | Quantity % (w/w) |
|---|---|---|
| 1 | Diclofenac sodium | 33.33 |
| 2 | Co-processed SR granulates of Example 1 | 66.66 |
| | Total | 100.00 |

Co-processed directly compressible slow release excipient prepared according to Example 1 of present invention having an average particle size of 200 to 400 microns is dry blended with diclofenac sodium in the ratio of 2:1 in octagonal blender for 10 minutes. This final mixture is compressed into tablets, (9 mm, round standard concave shape), each tablet containing 100 mg of Diclofenac sodium. The tablets weight was set at 300 mg. Results of dissolution test are shown in Table 5.

TABLE 5
Release profile of diclofenac sodium extended release tablets

| Time (hours) | Amount dissolved | Drug dissolved (%) |
|---|---|---|
| 1 | Between 15 and 35% | 18.56 |
| 5 | Between45 and65% | 49.47 |
| 10 | Between65 and85% | 72.94 |
| 16 | Between75 and95% | 96.36 |
| 24 | Not less than 80% | 100.03 |

### Example 4. Metformin Hydrochloride Extended Release Tablets—500 mg

TABLE 6
Formulation of Metformin Hydrochloride extended release tablets

| | | Quantity % | Quantity % |
|---|---|---|---|
| | | (w/w) Direct | (w/w) moist |
| | | Compression | granulation |
| Sr. No. | Name of Ingredients | Example 4a | Example 4b |
| 1 | Metformin Hydrochloride | 62.50 | 75.00 |
| 2 | Co-processed SR granulates | 37.50 | 25.00 |
| | of example 2 | | |
| | Total | 100.00 | 100.00 |

Co-processed directly compressible slow release excipient of Example 2 prepared according to present invention having an average particle size of 200 to 400 microns is blended with metformin hydrochloride and compressed into tablets. In example 4a, dry blending in octagonal blender in a ratio of 1:1.66, carried out for 5 min, followed by direct compression. Tablet specifications are shown in Table 7. In example 4b, excipient of example 2 is dry blended with metformin hydrochloride in octagonal blender in a ratio of 1:3 for 10 min. This dry blend is slightly wet granulated (moistened) using water as binder in rapid mixer granulator having impeller-chopper combination. Wet granulation is done in such way that final moisture content of granules is 4-5%. Drying step is not carried out in this experiment. These moistened granules were then compressed into tablets. Tablet specifications are shown in Table 7.

TABLE 7
Tablet specifications for Example 4a and Example 4b

| | | |
|---|---|---|
| Parameters | Example 4 a | Example 4 b |

| | | | | |
|---|---|---|---|---|
| Tablet weight | 800 | mg | 650 | mg |
| Tablet size | 14 × 8 | mm | 14 × 8 | mm |

| | | |
|---|---|---|
| Tablet shape | Standard caplet | Standard caplet |

Dissolution test carried out for tablets prepared according to example 4a and 4b. Results are shown in Table 8.

TABLE 8
Release Profile of Metformin Hydrochloride
extended release formulation

| Time (hours) | Amount dissolved | Example 4a | Example 4b |
|---|---|---|---|
| 1 | Between 20-40% | 35.01 | 32.57 |
| 2 | Between 35-55% | 46.11 | 48.38 |
| 5 | Between 60-80% | 73.18 | 72.21 |
| 12 | Not less than 80% | 93.65 | 95.74 |

### Example 5. Losartan Potassium Extended Release Tablets

Formulation were prepared using the process described in Example 3. Additionally formulations were also prepared using equivalent physical mixture of excipients employed in development of co-processed directly compressible granulates of Example 2. Composition of the formulation is shown in as per Table 9. Formulation quantities are expressed in weight %.

TABLE 9
Formulation of Losartan Potassium extended release tablet

| Ingredients | Quantity (w/w %) |
|---|---|
| Losartan potassium | 33.33 |
| Co-processed directly compressible | 65.16 |
| granulates of example 2 | |
| Mag. Stearate | 1 |
| Talc | 0.5 |
| Total | 100 |

Dissolution test carried out for tablets prepared according to Example 5. Results are shown in Table 10.

TABLE 10
Release Profile of Losartan Potassium extended release formulation

| | | Co-processed directly | Equivalent physical |
|---|---|---|---|
| | Amount | compressible granu- | mixture of granu- |
| Time | dissolved | lates of Example 2 | lates of Example 2 |
| (hours) | (%) | Drug dissolved (%) | Drug dissolved (%) |
| 0 | 0 | 0 | 0 |
| 2 | Between 15-25 | 19.60 | 25.91 |
| 4 | Between25-40 | 32.88 | 41.35 |
| 6 | Between40-60 | 51.61 | 72.84 |
| 8 | Between60-80 | 73.05 | 89.36 |
| 10 | Between85-110 | 92.02 | 102.48 |

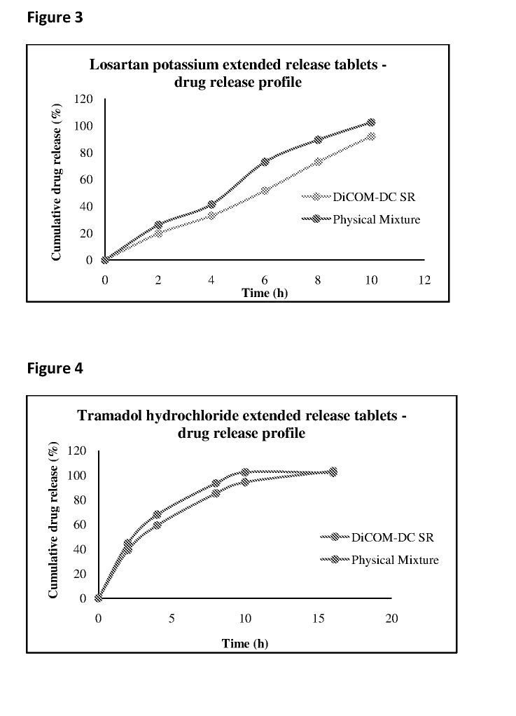

FIG. 3 provides the release profile of Losartan potassium extended release tablets.

### Example 6. Tramadol HCl Extended Release Tablets

Formulations were prepared using the process described in Example 3. Additionally formulations were also prepared using equivalent physical mixture of excipients employed in development of co-processed directly compressible granulates of Example 1. Composition of the formulation is shown in Table 11. Formulation quantities are expressed in weight %.

TABLE 11
Formulation of Tramadol Hydrochloride extended release tablet

| Ingredients | Quantity (w/w %) |
|---|---|
| Tramadol HCl | 33.33 |
| DiCOM-DC SR300 | 65.16 |
| Mag. Stearate | 1 |
| Talc | 0.5 |
| Total | 100 |

Dissolution test carried out for tablets prepared according to Example 6. Results are shown in Table 12.

TABLE 12
Release Profile of Tramadol HClextended release formulation

| | Co-processed directly | Equivalent physical |
|---|---|---|
| | compressible granu- | mixture of granu- |
| Time | lates of example 1 | lates of example 1 |
| (hours) | Drug dissolved (%) | Drug dissolved (%) |
| 0 | 0 | 0 |
| 2 | 39.13 | 44.63 |
| 4 | 59.23 | 67.84 |
| 8 | 85.20 | 93.37 |
| 10 | 94.31 | 102.34 |
| 16 | 103.33 | 101.82 |

FIG. 4, further provides the release profile of Tramadol hydrochloride extended release tablets.

Drug Release Profiles:

Drug release profile of all the above mentioned examples is given as cumulative percentage of drug released over time. Data for average of six tablets is shown. Data shows that the tablet formulation prepared using Co-processed granulates prepared using present invention gave improved and controlled drug release profile as compared to reference tablets (prepared using equivalent physical mixture of excipients). Tablets prepared using present invention has shown controlled and extended drug release as compared to reference tablets. Present invention has better release retarding potential as compared to equivalent simple physical mixture of same excipients.

We claim:

1. A process of producing a co-processed directly compressible excipient comprising an inert diluent, a hydrophilic swellable polymer and a binder, wherein the said compressible excipient controls the rate of drug release.

2. The process of producing a co-processed directly compressible excipient of claim 1, wherein the binder is selected from water, organic solvent, starch paste, hydrophilic polymer or a combination thereof.

3. The process of producing a co-processed directly compressible excipient of claim 1, wherein the diluent is carbohydrate or microcrystalline cellulose.

4. The process of producing a co-processed directly compressible excipient of claim 1, wherein the hydrophilic swellable polymer is a neutral polymer selected from the group consisting of synthetic polymers, cellulose based polymers, hydrocolloids and a combination thereof.

5. The process of producing a co-processed directly compressible excipient of claim 4, wherein the synthetic polymer is selected from the group consisting of poly acrylic acid derivatives, poly vinyl alcohol derivatives and a combination thereof.

6. The process of producing a co-processed directly compressible excipient of claim 4, wherein the cellulose based polymer is nonionic or substituted.

7. The process of producing a co-processed directly compressible excipient of claim 4, wherein the cellulose based polymer is selected from the group consisting of hypromellose, hypromellosepthalate, hydroxypropyl cellulose, hydroxyethyl cellulose, carboxymethylcellulose, carboxyethyl cellulose, ethyl cellulose, methyl cellulose, their salts and a combination thereof.

8. The process of producing a co-processed directly compressible excipient of claim 4, wherein the hydrocolloids is selected from the group consisting of gum arabic, guar gum, xanthan gum, alginic acid derivatives, carrageenan, chitosan and a combination thereof.

9. A process for producing a co-processed directly compressible excipient comprising the steps of:
a) mixing water insoluble diluent and water to form wet mass of diluent;
b) unloading wet mass of step (a) and collect in a collector;
c) performing dry blending by loading pre-sifted hydrophilic swellable polymer in high shear mixer granulator having impeller-chopper combination and dry mix;
d) slow addition of wet mass of diluent of step (a) to the dry powder blend of step (c) to granulate dry blend and addition of binder during granulation;
e) discharging wet granulated mass and drying it in fluidized bed dryer; and
f) addition of glidant and/or lubricants to the dried and sized granules.

10. The process of producing a co-processed directly compressible excipient of claim 9, wherein the said diluent is microcrystalline cellulose and the said binder is water.

11. The process of producing a co-processed directly compressible excipient of claim 9, wherein the said of glidant and/or lubricants is selected from the group consisting of stearic acid, magnesium stearate, calcium stearate, sodium stearate, boric acid, sodium lauryl sulfate, magnesium lauryl sulfate, corn starch, talcum, flow regulator and hardness enhancing agent
